(19) United States
(12) Patent Application Publication
(10) Pub. No.: US 2012/0283109 A1
(43) Pub. Date: Nov. 8, 2012

(54) METHOD OF ANALYZING TARGET NUCLEIC ACID OF BIOLOGICAL SAMPLES

(75) Inventor: Timothy Z. LIU, Fremont, CA (US)

(73) Assignee: Timothy Z. LIU, Fremont, CA (US)

(21) Appl. No.: 13/441,952

(22) Filed: Apr. 9, 2012

Related U.S. Application Data
(60) Provisional application No. 61/473,182, filed on Apr. 8, 2011.

(51) Int. Cl. C40B 20/04; C12Q 1/68

(52) U.S. Cl. 506/4; 435/6.11

(57) ABSTRACT

This invention provides a method of analyzing target nucleic acids of biological samples for multiplex nucleic acid analysis of disease associated genetic changes of biological samples in biomedical research and clinical diagnostics.

### RELATED APPLICATIONS

This application claims the benefit of, and priority to, U.S. Provisional Patent Application Ser. No. 61/473,182 filed on Apr. 8, 2011, the entire disclosure of which is hereby incorporated by reference in their entireties.

### SEQUENCE LISTING

The sequence listing submitted via EFS, in compliance with 37 CFR §1.52(e)(5), is incorporated herein by reference. The sequence listing text file submitted via EFS contains the file “TWT02141US-SeqListing.txt”, created on Apr. 9, 2012, which is 844 bytes in size.

### BACKGROUND

1. Field of Invention

This application is generally related to the detection and analysis of nucleic acid molecules from biological samples, and particularly to the determination of single nucleotide polymorphisms (SNPs), mutations, and other disease associated changes of target nucleic acid molecules.

2. Description of Related Art

Genomic DNA can be used in various clinical diagnostic applications, such as DNA sequencing, Single nucleotide polymorphism (SNP) genotyping, and mutation screening for associated diseases. The information from these tests can be used in disease predisposition screening, disease treatment stratification, and personalized medical management. With the completion of the human genome project a decade ago, the advent of personalized medicine offers a huge potential for future human healthcare and healthcare management. Genetic tests for individual, predisposition in disease prevention and management, and for personalized disease treatment, are under development, especially in the field of oncology. Successful examples of such genetic tests include SNP genotyping of variants in genes encoding and regulating Cytochrome P450 enzymes (e.g. CYP2D6, CYP2C19, and CYP2C9), which regulate the metabolization of various drugs, to improve drug response and reduce side-effects; and genetic mutation screening for disease-causing mutations in the BRCA1 and BRCA2 genes, which are implicated in familial breast and ovarian cancer syndromes. SNP genotyping is the analysis of single base pair mutation at specific loci that usually consisting of two alleles. It is one of the most common genetic variations that are associated with human diseases, and is widely used in biomedical research and clinical diagnostics.

Complex diseases such as cancers involve genetic changes of many genes. Many studies have revealed that panel testing of multiple biomarkers is a much better approach to accurately diagnose cancers, compared to current testing methods that only analyze a single or a few biomarkers, especially for non-invasive early cancer detection using clinical body fluid samples.

Current technologies used for medical genetic testing mainly include quantitative PCR, microarrays, and DNA sequencing. These technologies require extensive sample preparation of target DNA for purification and labeling.

Moreover, methods for nucleic acid analysis in clinical diagnostics normally include many steps, including DNA extraction and purification, amplification, and detection and quantification. Current methods of nucleic acid extraction and purification from biological samples require laborious and time consuming workflows, typically involving lysis of cells, precipitation of cell debris, followed by DNA precipitation and resuspension, using chemical solvents and centrifugation or vacuum in multiple steps and tubes. It has been a bottleneck for automated clinical molecular diagnostic systems.

### SUMMARY

In one aspect, the present invention provides a genetic analysis method of biological samples, which involves a simplified method of the detection and analysis of nucleic acid molecules from biological samples. In this disclosed method, target nucleic acid molecules from biological samples are isolated by hybridization on capture microparticles that contain target specific capture probes on the surface. Each capture microparticle contains at least one cluster of a target specific capture probes attached on its surface, wherein each target specific capture probe on the capture microparticle comprises an identity sequence (IS) tag that is assigned an identity code (ID code) and represents a predetermined gene of interest in an assay. The sequence variation of the aforementioned captured target nucleic acid molecules on the capture microparticles can be analyzed in parallel by various methods, such as labeled single base extension, or probe hybridization and ligation. Subsequently, the identity codes (ID codes) of the IS tags embedded in the target specific capture probes are determined on each capture microparticle from the assay. By mapping the identity codes of the IS tags on the capture microparticles to the sequence variation of the captured target nucleic acid molecules, multiple genes of interest can be simultaneously analyzed in a single multiplex assay. The number of target nucleic acid molecules that can be analyzed by this disclosed method can be easily scaled from a few to thousands in a single assay.

According one embodiment, the present invention provides a method of analysis of a target nucleic acid analyte. In this method, target nucleic acid molecules from biological samples, such as clinical blood samples, are isolated by contact and hybridization on capture microparticles that contain target specific capture probes on the surface. Each capture microparticle contains at least one cluster of target specific capture probes attached on its surface, wherein each target specific capture probe on the capture microparticle comprises an identity sequence (IS) tag that is assigned an identity code (ID code) and represents a predetermined target nucleic acid sequence of interest in an assay. The sequence variation of the aforementioned captured target nucleic acid molecules on the capture microparticles can be analyzed in parallel by various methods, such as labeled single base extension, or probe hybridization and ligation. Subsequently, the identity codes (ID codes) of the IS tags embedded in the target specific capture probes can be determined by in parallel by sequential paired-probe ligation chemistry on each capture microparticle from the assay. By mapping the identity codes of the IS tags on the capture microparticles to the sequence variation of the captured target nucleic acid molecules, multiple genes of interest can be simultaneously analyzed in a single multiplex assay. The number of target sequences that can be analyzed by this disclosed method can be easily scaled from a few to thousands in a single assay. With respect to the details of the sequential paired-probe ligation chemistry, it was described in U.S. patent application Ser. No. 13/252,095, which is herein incorporated by reference.

According another embodiment, the present invention provides a method of analysis of target nucleic acid sequences in a biological sample. In this disclosed method, single stranded target nucleic acid molecules from biological samples, such as clinical blood samples, are isolated by treating the sample with lysis buffer and lamda exonuclease before hybridization pullout on capture microparticles that contain target specific capture probes on the surface. The said capture microparticles contain at least one cluster of a target specific capture probe attached on its surface, wherein each target specific capture probe on the capture microparticle comprises an identity sequence (IS) tag that is assigned an identity code (ID code) and represents a predetermined target nucleic acid sequence of interest in an assay. The said capture microparticles are subjected to further analysis according to the method disclosed in this application.

According another embodiment, this invention provides a system that enables the disclosed method. In an embodiment, the system comprises a flow-cell where the captured target molecules can be analyzed, a thermal control unit that can regulate the temperature of parts of the flow-cell, a magnetic field control component that can apply a magnetic field to a surface in the flow-cell, a detector that can selectively detect signals from different labels, a fluid control system that can deliver reagents needed for carrying out the sample treatment and detection chemistry, and an electronic unit that controls the operation of the system and computes the results from the analysis. With respect to the detail of the system, it was described in U.S. patent application Ser. No. 13/252,095, which is herein incorporated by reference.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a schematic flow chart of a method of analysis of a target nucleic acid analyte according to an embodiment of the present invention.

FIG. 2 is an exemplary genotyping assay on capture microparticles according to an embodiment of the present invention.

FIG. 3 is an exemplary system for implementing the genotyping assay on capture microparticles according to an embodiment of the present invention.

FIGS. 4A to 4D are several images of OLA of VKORC1 on capture microparticles, in which each image is visualized by white light (FIG. 4A) or labeled by various dyes such as FAM (FIG. 4B), CY3 (FIG. 4C) and CY5 (FIG. 4D) under a microscope.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

In one aspect, the present invention provides a disclosed method and system used for multiplex analysis of target nucleic acid molecules in biomedical research and clinical diagnostics.

As used herein, “nucleic acid” or “oligonucleotide” or grammatical equivalents herein is meant at least two nucleotides covalently linked together. A nucleic acid of the present invention will generally contain phosphodiester bonds, although in some cases, for example when the primers contain labels, in nucleic acid analogs can be used. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine and hypoxathanine, etc. As used herein, the term “nucleoside” includes nucleotides and nucleoside and nucleotide analogs, and modified nucleosides such as label-modified nucleosides.

As used herein, “target nucleic acid molecule”, or “target nucleic acid sequence” or “target sequence” or grammatical equivalents herein means a nucleic acid sequence on a single strand or double strand of nucleic acid. The target sequence may be a portion of a gene, a regulatory sequence, genomic DNA, cDNA, RNA including mRNA and rRNA, or others. It may be any length, with the understanding that longer sequences are more specific. In some embodiments, it may be desirable to fragment or cleave the sample nucleic acid into fragments of 100 to 10,000 base pairs, with fragments of roughly 500 base pairs being preferred in some embodiments. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others.

As outlined herein, in some embodiments, the target sequence comprises a position for which sequence information is desired, generally referred to herein as the “detection position” or “detection locus”. In one embodiment, the detection position is a single nucleotide, although in some embodiments, it may comprise a plurality of nucleotides, either contiguous with each other or separated by one or more nucleotides.

By “plurality” as used herein is meant at least two.

As used herein, the base that base-pairs with a detection position base in a hybrid is termed a “target capture sequence” or an “hybridization pullout capture region”; thus many of the probes of the invention comprise the target capture sequence.

As used herein, a “single stranded target nucleic acid”, “single stranded target”, “single stranded target sequence” or grammatical equivalents thereof, is meant the starting material for the amplification methods of the present invention. In another embodiment, a target sequence of the present invention contains a region that is substantially complementary to a probe sequence, as defined herein.

As used herein, the sample comprising the target nucleic acid sequence may be virtually from any organism, and any sources, including, but not limited to, bodily fluids (including, but not limited to, blood, bone marrow, urine, feces, tears, serum, lymph, saliva, anal and vaginal secretions, perspiration, semen, and other bodily fluids of virtually any organism, such as mammalian, including human, samples); cell lysates of bacteria and pathogens, including viruses; hard tissues (e.g. organs such as liver, spleen, kidney, heart, lung, etc.); environmental samples (including, but not limited to, air, agricultural, water and soil samples); biological warfare agent samples; research samples (i.e. the sample may be the products of an amplification reaction, including both target and signal amplification as is generally described such as a PCR amplification reaction); purified samples, such as purified genomic DNA, RNA, proteins, etc.; raw samples (bacteria, virus, genomic DNA, etc.); as will be appreciated by those in the art, virtually any experimental manipulation may have been done on the sample.

If required, the target sequence is prepared using known techniques. For example, the sample may be treated to lyze the cells, using known lysis buffers, electroporation, etc., with purification and/or amplification occurring as needed, as will be appreciated by those in the art.

In some embodiments, a first template molecule and a second template molecule are generated from target nucleic acid molecules through an enzymatic reaction, using primers comprising sequences complimentary to target specific capture probe sequences and sequences that are complimentary to at least part of the target nucleic acid sequences, wherein the quantity ratios of the two target nucleic acid sequences in the sample are preserved.

In some embodiments, the first and the second target nucleic acid sequences are similar in abundance. In some embodiments, the first target nucleic acid sequence is at least 100, 1000, or 10,000 times more abundant than the second target nucleic acid sequence.

In some embodiments, the method of the invention comprises the use of target specific capture probes, whose sequences comprise an identity sequence (IS) tag and sequences that are complimentary to part of target nucleic acid sequences.

By “identity sequence (IS) tag” herein is meant a short artificial DNA sequence that is used to encode a target molecule, as a way for identifying a specific target sequence among analytes in a sample. In some embodiments, the IS tag is less than, 10 bases, or less than 6 bases in length. IS tags may also comprise additional nucleic acid sequences that are needed for probe hybridization during decoding process. IS tags are generally designed to be unique from the sequence of the genome of interest in nucleic acid analysis. In some embodiments, said IS tags are introduced as part of target specific capture probe preparation.

By “identity (ID) code” herein is meant a code assigned to an IS tag. The base composition of each IS tag corresponds to a specific ID code.

By “located spatially separately” herein is meant that two or more clusters of target specific capture probes are located separately in space. For example, the different clusters of target specific capture probes can locate on different spots on the same surface (e.g. a continuous surface), or locate on different surface, such as on the surface of different capture microparticles as described herein.

In some embodiments, the clusters of target specific capture probes are attached on a surface. The target specific capture probes are attached to the surface directly or indirectly. In some embodiments, the target specific capture probes are attached to the surface of a capture microparticle (i.e. a paramagnetic microparticle), and the capture microparticle is immobilized on the surface by a physical force (e.g. magnetic field) or by a chemical linkage described herein or known in the art.

In some embodiments, the ID codes of the IS tags are identified to determine the target nucleic acid sequences as described herein.

By “sequence variation” herein is meant the characteristics of a sequence, such as single nucleotide polymorphism (SNP), mutation, or methylation. Sequence variation can be determined by the methods known in the art or disclosed herein, including, but not limited to labeled probe ligation, single-base extension, DNA sequencing, and melting curve analysis.

As used herein, the term “single nucleotide polymorphism” or “SNP” refers to any position along a nucleotide sequence that has one or more variant nucleotides. Single nucleotide polymorphisms (SNPs) are the most common form of DNA sequence variation found in the human genome and are generally defined as a difference from the baseline reference DNA sequence which has been produced as part of the Human Genome Project or as a difference found between a subset of individuals drawn from the population at large, SNPs occur at an average rate of approximately 1 SNP/1000 base pairs when comparing any two randomly chosen human chromosomes. Extremely rare SNPs can be identified which may be restricted to a specific individual or family, or conversely can be found to be extremely common in the general population (present in many unrelated individuals). SNPs can arise due to errors in DNA replication (i.e., spontaneously) or due to mutagenic agents (i.e., from a specific DNA damaging material) and can be transmitted during reproduction of the organism to subsequent generations of individuals.

Hereinafter, a method for the hybridization pullout, optional amplification, and analysis of target nucleic acid molecules from biological samples is disclosed.

### Target DNA Sequence Extraction and Isolation Using Lambda Exonuclease

Exonucleases are enzymes that cleave nucleotides one by one at the end of a polynucleotide chain by hydrolysis of phosphodiester bonds. Some of the exonuleases work from 3′ to 5′ direction, and the others work from 5′ to 3′ direction. Lambda exonuclease is a highly processive 5′ to 3′ exonuclease that to digests the 5′ phosphorylated strand of double strand DNA (dsDNA), producing single strand DNA (ssDNA) fragments. Application of lambda exonuclease for ssDNA generation can be found in Avci-Adali M. et al “Upgrading SELEX Technology by Using Lambda Exonuclease Digestion for Single-Stranded DNA Generation”, Molecules 15:1-11, 2010; which is herein incorporated by reference. In contrast, it has reduced activity against single strand DNA (ssDNA). Exonuclease has been used in ssDNA generation from PCR products in various applications, such as DNA sequencing and microarrays.

Human blood samples are frequently used in clinical diagnostics. The leukocytes in human blood that contain genomic DNA (gDNA) comprise only 1% to 2% of all blood cells. About 3 μg genomic DNA can normally be isolated from 1 mL of whole blood sample using commercially available DNA extraction methods, corresponding to about 1×10⁷ to 10⁸ copies of gDNA. It is possible for certain sensitive nucleic acid diagnostic applications using blood samples without further PCR amplification.

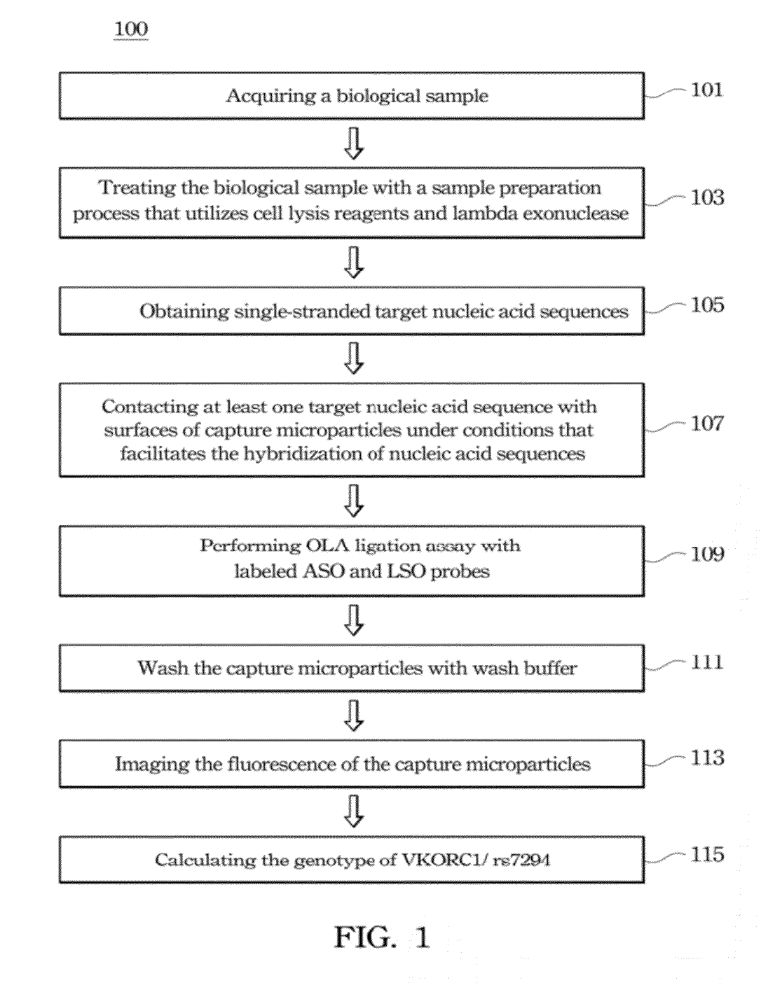

In this disclosed method, the cell lysis and DNA isolation by hybridization pullout on capture microparticles are combined in a continuous process in a single reaction vessel. Lambda exonulcease can be used in the process in order to generate single strand DNA fragments for selective hybridization pullout of target nucleic acid sequences from blood samples. The target nucleic acid sequences can be effectively isolated on the capture microparticles using this method as summarized in FIG. 1.

Reference is made to FIG. 1, which is a schematic flow chart of a method of analysis of a target nucleic acid analyte according to an embodiment of the present invention. The following method 100 is an exemplary DNA extraction and isolation for SNP genotyping from blood samples, which comprises the following steps:

Step 101. Acquire a biological sample. In an embodiment, the biological sample may be a human blood sample. However, in other embodiments, the biological sample may be other types of samples.

Step 103 and Step 105. Treat the biological sample with a sample preparation process that utilizes cell lysis reagents and lambda exo-nuclease for obtaining single-stranded target nucleic acid sequences. Add the biological sample (e.g. human blood sample) into a sample treatment buffer mix in a reaction vessel. In an embodiment, the sample treatment buffer mix including lysis reagents and lambda exonuclease can treat the biological sample simultaneously for obtaining single-stranded target nucleic acid sequences. Single-strand genomic DNA (gDNA) fragments (or called “single-stranded target nucleic acid sequences” or “target ssDNA sequences”) are randomly generated by the 5′ to 3′ nuclease activity of the lambda exonuclease enzyme.

The aforementioned lysis reagent further includes NaOH, tween 80, EDTA, PEG, lauryl-sarcosine salt, and optionally proteinase K. Incubate the reaction mixture at room temperature or 37° C. for a period of time, normally for less than 10 minutes, so as to lyze the blood sample. The pH of this sample lysis mixture can be optionally adjusted before ensuing steps.

In other embodiment, after lyzing the biological sample with the cell lysis reagents, the biological sample is further treated by lambda exonuclease is then added into the same reaction vessel for obtaining single-stranded target nucleic acid sequences. After adding the lambda exonuclease enzyme and its related buffer mix into the same reaction vessel, incubate this reaction mix at 37° C. for a certain period of time, normally for less than 30 minutes.

Step 107. Contacting at least one single-stranded target nucleic acid sequence with surfaces of capture microparticles under conditions that facilitates the hybridization of nucleic acid sequences. Add capture microparticles and hybridization buffer into the reaction mix from Step 105 in the same reaction vessel. The pH of the mixture after the hybridization buffer addition is maintained within a range that nucleic acid hybridization can occur. The surface of each capture microparticle comprises at least one cluster of target specific capture probes. Each target specific capture probe comprises an IS tag and a sequence that is complimentary to part of a target nucleic acid sequence of interest. Each cluster of the target specific capture probe is spatially separated from another cluster and their relative location is fixed in relation to each other.

These capture microparticles can be paramagnetic microparticles. There should be as many types of capture microparticles as the number of target nucleic acid sequences of interest in the SNP genotyping assay. This mixture is allowed to mix well and incubate for a specified period of time, normally less than one hour. The temperature of the container can be optionally regulated during this period of time. Selected target DNA fragments are thus retained on the capture microparticles by hybridization pullout.

The ratio of different types of capture microparticles can be the same or different for said different target nucleic acid sequences, depending on the to assay design, for quantitation purpose.

Optionally, before hybridizing (or contacting) the target nucleic acid sequences with the capture microparticles, a plurality of single stranded target nucleic acid sequences can be generated from the treated biological sample in step 105 with a nucleic acid asymmetric amplifying method such as asymmetric PCR.

### Multiplex Nucleic Acid Analysis Using Identity Sequence Tags

The captured target DNA fragments on the remaining paramagnetic microparticles from Step 107 can be subjected to further sequence analysis for genetic changes.

Step 109. Perform OLA ligation assay with labeled ASO and LSO probes.

All the paramagnetic capture microparticles in the reaction vessel during Step 107 are randomly distributed and held in position on the surface of a flow-cell, i.e. the location of each of the capture microparticles is known in relation to each other, throughout the ensuing analysis process. The nucleic acid sequence at specified loci of genes of interest that are contained in the DNA fragments pulled out from the sample can be analyzed by various methods, such as labeled oligonucleotide probe hybridization and ligation on the capture microparticles in the flow-cell. The labels on the oligonucleotide probes can be different fluorescent dyes.

For example, in SNP genotyping, one color can represent a specific allele, while another color can represent the other specific allele. The capture microparticles can be imaged on the surface. However, the same set of dyes can be used for all target loci of interest in a single assay in this invention. Each microparticle will only test a specific locus of interest, and the micrparticles are spatially separated from other microparticles.

The label information obtained on said capture microparticles in step 109 will be used in conjunction with ID codes obtained in step 113.

Step 111. Wash the capture microparticles with wash buffer. After the aforementioned sequence analysis by labeled probes, the captured DNA fragments can be dissociated from the paramagnetic capture microparticles under denaturing conditions, such as in high pH conditions or with chaotropic agents, e.g. guanidine, which leaves only target specific capture probes on the paramagnetic microparticles. The remaining capture microparticles can be washed with the wash buffer in the flow-cell, wherein the locations of these microparticles are kept and fixed with respect to each other.

Step 113. Image the fluorescence of the capture microparticles on the surface. The IS tags embedded in the target specific capture probes attached on the surface of these microparticles can then be determined by sequential paired-probe ligation chemistry disclosed in patent application W0/2010/115100A1, which is herein incorporated by reference. In brief, the sequential paired-probe ligation chemistry can be carried out with pools of labeled oligonucleotide probes, including sets of 5′ labeled IS probes (5′_LISPs) and 3′ labeled IS probes (3′-LISPs) in the same flow-cell. With respect to the sequential paired-probe ligation chemistry, it is also described in U.S. patent application Ser. No. 13/252,095, which is herein incorporated by reference.

The color representation obtained from said labels on each capture microparticle in step 109, such as SNP alleles and mutation variation, can be to combined with the identity codes from the IS (identity sequence) tags embedded in the target specific capture probes attached on said capture microparticles in step 113 to reveal the genetic variation of each target nucleic acid sequence of interest. Those skilled in the art can easily appreciate the association between the ID codes and the genetic variation information derived from each capture microparticle in the assay. With respect to the identity codes and the IS tags embedded in the target specific capture probes, they are described in U.S. patent application Ser. No. 13/252,095, which is herein incorporated by reference.

The identity codes represented by the IS tags on the capture microparticles are assigned to specified genes of interest in the assay design, which can be easily scaled from a few to thousands in a single assay in this disclosed method.

Step 115. Calculate the genotype of VKORC1/rs7294. The sequence variation of at least one target nucleic acid sequence that has been hybridized to said target specific capture probe on said surface is further analyzed as follows. And then, the identity code of said IS tag embedded in said target specific capture probe on said surface are determined. Subsequently, these said clusters can be labeled as “positive” clusters, which can be identified by the probe labels used in the sequence variation analysis step, for example, step 109 in FIG. 1. The amount of each target nucleic acid sequence in the sample is directly proportional to the number of “positive” clusters of respective target specific capture probes. By comparing the amount of “positive” clusters of each said target specific capture probes, the relative abundance of said target nucleic acid sequences in the analyte can be to calculated. Calibration curves can also be used for the quantitation.

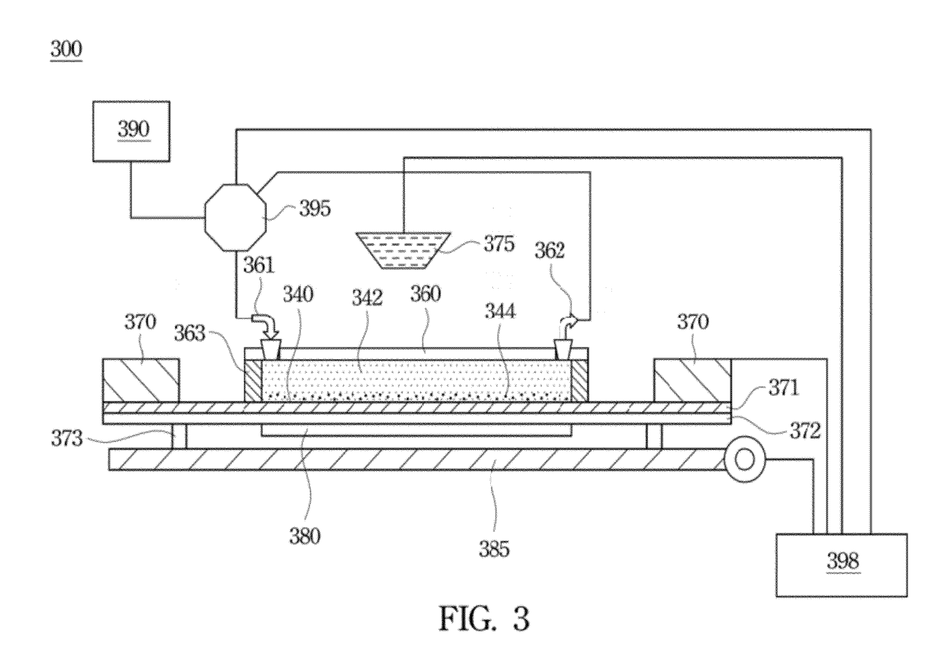

In other embodiments, the aforementioned method can be implemented in a system illustrated in FIG. 3. The system 300 comprises a flow-cell 363 where the captured target molecules can be analyzed. A thermal control unit 371 disposed under the flow-cell 363 can regulate the temperature of parts of the flow-cell 363. The thermal control unit 371 includes one or optionally two thermal electric heating and cooling units 370 attached for regulating the temperature of the reaction surface inside the flow-cell, and a thermal isolation layer 372 optionally placed between the thermal conducting plate 371 and supported by stands 373. A magnetic field control component 380 can apply a magnetic field to a surface in the flow-cell 363.

The whole assembly is mounted on an x-y precision moving stage 385 that can accommodate the scan area of the detection window 360 in the flow-cell 363. A detection unit 375 is mounted directly facing the detection window 360 of the flow-cell 363, and is capable of automatically maintaining the focus and selectively detect signals from different labels in the assay. Methods of fluorescence imaging are well known in the art. An example is a fluorescence microscope with filter cubes for different excitation and emission spectra. Additionally, the detection unit 375 also comprises a CCD imaging camera (unshown).

A fluidic system 395 is connected to a reagent unit 390 where all necessary reagents needed for carrying out the sample treatment and detection chemistry in the assay are stored, and optionally kept at specified temperature. The fluidic system 395 controls the delivery and removal of reagents from the inlet 361 and outlet 362 of the flow-cell 363, as well as waste control. All the control and data processing are handled by a electronic unit 398 that controls the operation of the system, processes data and computes the results from the analysis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

### EXAMPLES

### Example 1

### SNP Analysis for Personalized Medicine

Warfarin is the most commonly prescribed anticoagulant for the treatment and prevention of arterial and venous thromboembolism. Warfarin has a narrow therapeutic dosage range for different patients, for which various SNPs, including VKORC1, are known to cause warfarin sensitivity, and account for 35 to 50% of the variability in warfarin dosage requirement. More detailed information about warfarin sensitivity can be found in Chen, et al. “Genetic Variants Predicting Warfarin Sensitivity”, US 2011/0236885 A1; which is incorporated by reference. Moreover, FDA approved the updated labeling for warfarin in 2007, which highlights the opportunity for healthcare providers to use genetic tests to improve the initial drug dosage estimate for individual patients (FDA, http://www.fda.gov/NewsEvents/Newsroom/PressAnnouncements/2007/ucm108967.htm).

In this example, the locus of VKORC1/rs7294 was selected as the target nucleic acid sequence for SNP genotyping by the disclosed method. The reference sequences of VKORC1/rs7294 SNP locus was obtained from NCBI SNP database available on the internet (http://vvww.ncbi.nlm.nih.gov/projects/SNP/).

Oligonucleotide ligation assay (OLA) is a specific and sensitive method for the determination of single nucleotide polymorphism (SNP). Specifically, OLA method is based on the ligation of two adjacent oligonucleotide probes by a ligase at the SNP site of the target loci. In OLA assays, there is one common oligonucleotide probe called locus specific probe (LSO) which is complementary to the target DNA template sequence to one side of the SNP site, and there are two labeled reporter oligonucleotide probes called allele specific probes (ASOs) that comprise one of the two possible complementary bases at the ligation site. The labels on the two allele specific probes, ASOs, can be two distinctive fluorescent dyes. Only the ASO probe that contains the complementary base at the SNP site of the target nucleic acid sequence will be ligated by a DNA ligase. The presence of the ligated product reveals the allele variation of the target DNA sequence indicated by the ligated ASO label. The specificity of OLA is conferred by the ligase enzyme used in the assay.

In this example, the VKORC1/rs7294 SNPs are A/G alleles. A set of four oligonucleotide probes are synthesized for each SNP locus: one 5′ biotinylated target specific capture probe (SEQ ID No. 1), one locus specific oligonucleotide (LSO) probe (SEQ ID No. 2), that is both 5′ phosphorylated and 3′ FAM labeled; and two allele specific oligonucleotides (ASO) probes that are labeled by cyanines either Cy3 or Cy5 dyes at the 5′ ends, SEQ ID No. 3 and SEQ ID No. 4, representing either A allele or G allele respectively of the target VKORC1/rs7294 sequence. Unlike previously reported LSO in OLA that is not labeled, the LSO in this disclosed method is labeled to serve as an indicator of the ligation chemistry.

The oligonucleotide probe sequences used for analyzing VKORC1/rs7294 in this example are as follows,

TABLE 1

| | | SEQ ID |
|---|---|---|
| Name | Sequence | NO. |
| 5′ biotinylated capture probe | 5′ biotin-c12-CTT TGG AGA CCA GCC CAT GGG GAC AGA GTC AGA 3′ | 1 |
| locus specific oligonucleotide | 5′ Phos-GGG TAT GGC AGG AGG AGG-FAM 3′ | 2 |
| (LSO) probe | | |
| allele specific oligonucleotides | 5′ Cy3-CAC ATT TGG TCC ATT GTC ATG TGT 3′ | 3 |
| (ASO) probes | 5′ Cy5-ACA TTT GGT CCA TTG TCA TGT GC 3′ | 4 |

For VKORC1/rs7294 fragment, the target specific capture probe is a 5′ biotinylated oligonucleotide of 33 bases long (SEQ ID NO. 1) that is about 55 bases away from the SNP site. The target specific capture probes were attached to streptavidin coated paramagnetic microparticles before being used in the SNP genotyping assay.

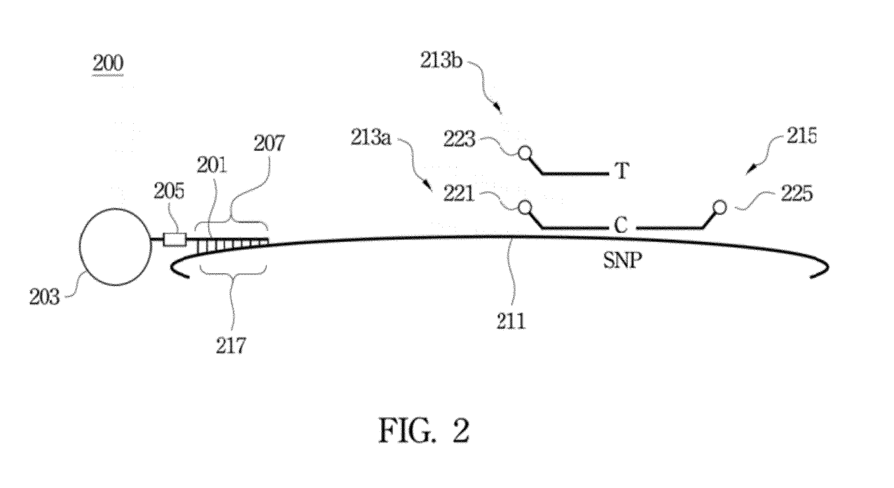

A schematic representation of the exemplary genotyping assay of VKORC1 SNPs on the capture microparticles is shown in FIG. 2. In this exemplary method, the capture microparticle 203 is a paramagnetic microparticle of 1 μm in diameter. The sequence of the target specific capture probe 201 has an IS tag 205 embedded therein near the surface of the capture microparticle 203, and a target capture sequence 207 that is hybridized to the hybridization pullout capture region 217 of the target genomic DNA fragment 211. The target specific capture probes 201 for both SNPs were the same to ensure efficient pullout for both fragments.

Human blood samples were used as the source of genomic DNA for this example. The target genomic DNA fragments 211 from the blood samples were isolation on the capture microparticles 203 using aforementioned sample treatment method with lysis reagent and lambda exonuclease, without PCR amplification. This was followed by contacting the treated sample from above with the capture microparticles 203 that have the 5′ biotinylated target specific capture probe 201 attached on the surface and let its target capture sequence region 207 hybridize to pull out the DNA fragments 211 that contain the hybridization pullout capture region 217 of the VKORC1 SNP locus. OLA was then performed on these capture microparticles 203 by adding an ASO probe 213a, another ASO probe 213b and a LSO probe 215, in which the ASO probe 213a had one possible complementary base C at the SNP ligation site and labeled by a dye 221 (for example, Cy5 dye), the ASO probe 213b had another possible complementary base T at the SNP ligation site and labeled by a dye 223 (for example, Cy3 dye), and the LSO probe 215 had a complementary sequence at the SNP ligation site and labeled by a still another dye 225 (for example, FAM dye). The VKORC1 SNP alleles were determined by imaging these capture microparticles 203 with OLA probes (including the ASO probe 213a, the ASO probe 213b and the LSO probe 215) ligated on them, and analyzing the color signals of each capture microparticle 203. This exemplary assay method is summarized according to FIG. 1.

The assay method includes following steps:

Step 101. Acquire a biological sample. For example, the biological sample may be human blood sample volume of 25 μl.

Step 103 and Step 105. Treat the biological sample with a sample preparation process that utilizes cell lysis reagents and lambda exo-nuclease for obtaining single-stranded target nucleic acid sequences. For example, lyze to the blood sample by adding 5 μL of lysis buffer, and incubate it at 37° C. for 10 minutes. And then, the blood sample is further treated by lambda exonuclease and buffer, and incubated in 35 μL at 37° C. for 20 minutes, so as to generate single-strand genomic DNA (gDNA) fragments.

Step 107. Contacting at least one single-stranded target nucleic acid sequence with surfaces of capture microparticles under conditions that facilitates the hybridization of nucleic acid sequences. The surface of each capture microparticle comprises at least one cluster of target specific capture probes, and each cluster of target specific capture probes is spatially separated from another cluster and their relative location is fixed in relation to each other. For example, hybridization pullout of target ssDNA fragment is carried out by adding hybridization buffer, capture microparticles with biotinylated target specific capture probe (SEQ ID NO. 1) attached on the surface, labeled LSO probe (SEQ ID NO. 2) with FAM dye, and labeled ASO probes (SEQ ID Nos. 3 & 4) with Cy3 and Cy5 dyes in a total volume of 40 μL. And incubate the hybridization mixture at 65° C. for 1 minute and subsequently at 45° C. for 19 minutes.

Before contacting said target nucleic acid sequences with surfaces of capture microparticles in the step 107, a plurality of single stranded target nucleic acid sequences from said treated biological sample are optionally generated from the step 107, with an asymmetric amplifying method of said target nucleic acid sequences.

Step 109. Perform OLA ligation assay with labeled ASO and LSO probes, in which the OLA ligation reaction is initiated by adding ligation buffer to and T4 ligase in total 50 μL. Let it incubate at 25° C. for 20 minutes.

Step 111. Wash the capture microparticles with wash buffer, 2 to 3 times, at 60° C.

Step 113 and Step 115. Image the fluorescence of the capture microparticles under a fluorescence microscope, and analyze the fluorescent images in order to calculate the VKORC1/rs7294 SNP alleles of the target fragments according to the color map of each capture microparticle.

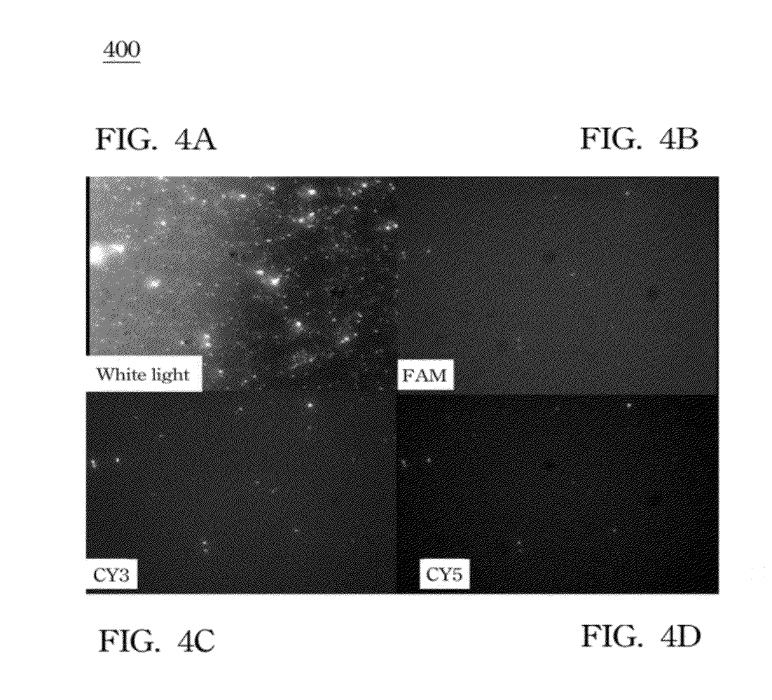

The LSO and ASO probes are specifically designed for each SNP locus according to OLA principles. The FAM label on the LSO probes is used to monitor the ligation reactions. The ligation chemistry was carried out with T4 DNA ligase. The capture microparticles from the OLA were washed under stringent conditions to remove unligated LSO and ASO probes. The remaining microparticles were subsequently imaged on a glass slide under a fluorescence microscope. Selective spots of the randomly distributed capture microparticles were imaged through 4 color channels, i.e. white light, Cy3, Cy5, and FAM. Exemplary images of VKORC1 capture microparticles after the OLA experiment were shown in FIGS. 4A to 4D. Each image in FIGS. 4A to FIG. 4D is labeled by the color channel through which it was taken. The capture microparticles that have FAM signals also gave Cy3 and Cy5 signals.

Since FAM signal is from the label of LSO, its presence indicates that these capture microparticles contain VKORC1 DNA fragments from the sample. The coexistence of Cy3 and Cy5 signals on these capture microparticles indicates that both SNP alleles of VKORC1 were in the DNA fragments from the blood sample, i.e. the blood sample tested in the assay is heterozygous for VOKRC1/rs7294 SNPs, in this example.

By knowing the genotypes of VKORC1, the healthcare professionals will be in a better position to tailor the dosages of warfarin for individual patients.

The IS tags embedded in the target specific capture probes can subsequently be determined by various methods, including pair-probe ligation chemistry, when there are more than one type of capture microparticles used in the assay. By correlating the identity codes of the IS tags to the sequence variation obtained on each microparticle, multiple genes can be simultaneously analyzed in a single assay.

Although the present invention has been described in considerable detail with reference to certain embodiments thereof, other embodiments are possible. For example, by designing various ASO probes and LSO probes, or by using many clusters of target specific capture probes located separately in space, or by labeling those probes with other fluorescent dyes, or implementing in other systems (e.g. arrays), the present method and system can be applied to analyze SNP genotyping on other genes such as CYP2D6, CYP2C19, CYP2C9 and so on. By correlating the identity codes of the IS tags to the sequence variation of the genes of interest, multiple genes can be simultaneously analyzed in a single assay even if the same dyes are used as labels on different sets of ASO probes. Therefore, their spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this its invention provided they fall within the scope of the following claims.

What is claimed is:

1. A method of analysis of a target nucleic acid analyte, comprising:
a. contacting said nucleic acid analyte comprising at least one target nucleic acid sequence with a surface under conditions that facilitates the hybridization of nucleic acid sequences, wherein said surface comprises at least one cluster of target specific capture probes, and wherein each said cluster of target specific capture probes is spatially separated from another cluster and their relative location is fixed in relation to each other, and each said target specific capture probe further comprises:
(i). a nucleic acid sequence that is complimentary to a portion of said target nucleic acid sequence in the said nucleic acid analyte; and
(ii). an identity sequence (IS) tag that is assigned an identity code that represents a predetermined target nucleic acid sequence;
b. analyzing at least one sequence variation of at least one said target nucleic acid sequence that is hybridized to said target specific capture probe on said surface;
c. determining the identity code of said identity sequence tag embedded in said target specific capture probe on said surface; and
d. mapping said sequence variations obtained in the step b to said identity codes of said target specific capture probes determined in the step c to derive the sequence variation of at least one target nucleic acid sequence.

2. The method of claim 1, wherein different clusters of target specific capture probes are attached to a continuous surface and spatially separated.

3. The method of claim 1, wherein different clusters of target specific capture probes are attached to the surface of different microparticles.

4. The method of claim 1, wherein each said cluster of target specific capture probes comprises the same IS tag and the same target specific capture nucleic acid sequence.

5. The method of claim 1, wherein said sequence variation of said target nucleic acid sequence can be analyzed by probe hybridization, probe ligation, single nucleotide extension, or DNA sequencing.

6. The method of claim 1, wherein said identity code of said target specific capture probes can be elucidated by DNA sequencing or sequential paired-probe ligation.

7. The method of claim 1, wherein the step of mapping said sequence variations further comprises:
computing the number of clusters of each target specific capture probe that contains the said target nucleic acid sequence, so as to quantify at least two target nucleic acid sequences.

8. A method of analysis of target nucleic acid sequences in a biological sample, comprising:
a. obtaining a plurality of single stranded target nucleic acid sequences from said biological sample treated by a sample preparation process that utilizes lambda exo-nuclease;
b. contacting said target nucleic acid sequences with a surface, under conditions that facilitates the hybridization of nucleic acid sequences, where said surface comprising at least one cluster of target specific capture probes, and wherein each said cluster of target specific capture probes is spatially separated from another cluster and their relative location is fixed in relation to each other, and each said target specific capture probe further comprises:
(i).a nucleic acid sequence that is complimentary to a portion of said target nucleic acid sequence in the said nucleic acid analyte, and
(ii).an identity sequence tag that corresponds to an identity code that is assigned to a predetermined target nucleic acid sequence,
c. analyzing at least one sequence variation of at least one said target nucleic acid sequence that is hybridized to said target specific capture probe on said surface;
d. determining the identity code of said identity sequence tag embedded in said target specific capture probes on said surface; and
e. mapping said sequence variations obtained in the step c to said identity code of said target specific capture probes determined in the step d to derive the sequence variation of at least one target nucleic acid sequence.

9. The method of claim 8, before contacting said target nucleic acid sequences with the surface in the step b, further comprising:
generating a plurality of single stranded target nucleic acid sequences from said treated biological sample from the step a, with an asymmetric amplifying method of said target nucleic acid sequences.


SEQUENCE LISTING (4 sequences)

SEQ ID NO: 1 - DNA, 33 bp, Artificial sequence
  Chemically synthesized sequence
ctttggagaccagcccatggggacagagtcaga

SEQ ID NO: 2 - DNA, 18 bp, Artificial sequence
  Chemically synthesized sequence
gggtatggcaggaggagg

SEQ ID NO: 3 - DNA, 24 bp, Artificial sequence
  Chemically synthesized sequence
cacatttggtccattgtcatgtgt

SEQ ID NO: 4 - DNA, 23 bp, Artificial sequence
  Chemically synthesized sequence
acatttggtccattgtcatgtgc